(19) United States
(12) Patent Application Publication
(10) Pub. No.: US 2026/0363978 A1
(43) Pub. Date: Nov. 19, 2026

(54) A TRANSPARENT LIQUID CLEANSING COMPOSITION

(71) Applicant: Conopco, Inc., d/b/a UNILEVER, Hoboken, NJ (US)

(72) Inventors: Shajahan ABDUL KAREEM, Pondicherrry (IN); Saswati PUJARI, Bangalore (IN)

(73) Assignee: Conopco, Inc., d/b/a UNILEVER, Hoboken, NJ (US)

(21) Appl. No.: 19/135,061

(22) PCT Filed: Dec. 1, 2023
(86) PCT No.: PCT/EP2023/083884

§ 371 Date: Jun. 2, 2025

(30) Foreign Application Priority Data
Dec. 8, 2022 (EP) 22212173.3

(51) Int. Cl. A61K 8/36; A61K 8/44; A61K 8/46; A61Q 19/10

(52) U.S. Cl. CPC A61K 8/361; A61K 8/442; A61K 8/463; A61Q 19/10; A61K 2800/262; A61K 2800/596

(57) ABSTRACT

The present invention relates to a liquid cleansing composition that comprises a soap and an amphoteric surfactant in select weight ratio along with select non-soap anionic surfactant. The transparency of the composition is less than 100 NTU when measured at temperatures from 4 to 25 °C and the viscosity is in the range from 2,500 to 10,000 cps measured at 25 °C with the composition delivering superior foam.

### FIELD OF THE INVENTION

The present invention relates to a transparent liquid cleansing composition. Particularly, the present invention relates to a liquid cleansing composition that is transparent at low temperatures e.g. 4 to 25 °C.

### BACKGROUND OF THE INVENTION

WO11057909 (Unilever) discloses a mild, substantially isotropic skin cleansing solution was found to be able to suspend insoluble components and provide copious amounts of lather. The cleanser is formulated with synthetic anionic surfactants and a specific ratio of carboxylic acid(s) to hydrophobically modified cross-linked acrylate copolymer(s). The carboxylic acid and acrylate polymer combination were found to provide a synergistic effect on zero shear viscosity at 25 °C in a specific pH and copolymer/acid concentration ratio range.

IN377416 (ITC Limited) discloses a transparent liquid cleanser composition. Particularly, IN377416 discloses a transparent liquid cleanser composition comprising (a) at least one free fatty acid; and (b) at least one anionic surfactant; and a method of preparing said composition.

FR3065874 discloses a cosmetic composition in the form of a liquid, clear hygiene product having a pH less than 8, characterized in that it comprises as active principle: —between 1% and 10% by mass of at least one fatty acid, —between 0.5% and 8% by mass of at least one fatty acid neutralizer reacting with the fatty acid so as to form soap, —between 1% and 25% by mass of at least one amphoteric primary surfactant belonging to the betaine family, —between 0.5 and 6% by mass of at least one amphoteric and/or anionic secondary surfactant, the other ingredients of the composition being constituted by those admissible in the field cosmetic.

Despite efforts thus far, delivering a liquid cleansing composition that is transparent at lower temperatures, e.g. from 4 to 25 °C, remains a challenge. Additionally, delivering such transparent compositions in presence of soap; and therefore in alkaline pH range, and yet having sufficient lathering adds to the challenge. Further, providing such transparent compositions with appropriate viscosity e.g. 2500 to 10,000 cps also adds to the challenge.

It has been found that when a composition comprises a soap and an amphoteric surfactant in select weight ratio, along with select anionic surfactant, the composition exhibited transparency of less than 100 nephelometric turbidity units (NTU) at lower temperature e.g. from 4 to 25 °C.

The composition exhibited viscosity in the range from 2,500 to 10,000 cps when measured at 25 °C. Excellent lathering as evidenced with high foaming was also obtained.

### SUMMARY OF THE INVENTION

The present invention relates to a transparent liquid cleansing composition comprising:
a. a soap; and
b. an amphoteric surfactant,
   wherein the soap and the amphoteric surfactant are present in the weight ratio less than or equal to 2:1,
   wherein the transparency of the composition is less than 100 NTU when measured at temperature from 4 to 25 °C; and
   wherein viscosity of the composition is in the range from 2,500 to 10,000 cps when measured at 25 °C;
   wherein the composition further comprises 1 to 25 wt % non-soap anionic surfactant selected from alkyl sulfate, alkyl ether sulfate, alpha olefin sulfonate and mixtures thereof.

### DETAILED DESCRIPTION OF THE INVENTION

Any feature of one aspect of the present invention may be utilized in any other aspect of the invention. The word “comprising” is intended to mean “including” but not necessarily “consisting of” or “composed of.” In other words, the listed steps or options need not be exhaustive. Except in the operating and comparative examples, or where otherwise explicitly indicated, all numbers in this description indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated. Unless specified otherwise, amounts as used herein are expressed in percentage by weight based on total weight of the composition and may be abbreviated as “wt %”. The use of any and all examples or exemplary language e.g. “such as” provided herein is intended merely to better illuminate the invention and does not in any way limit the scope of the invention otherwise claimed. The terms ‘Skin’ as used herein, preferably means to include skin on any part of the body e.g. face, neck, chest, back, arms, underarms, hands, legs, buttocks and scalp.

The term ‘transparent’ as used herein preferably means the transparency of the liquid composition is less than 100, preferably less than 75, more preferably less than 50, even more preferably less than 25, nephelometric turbidity units (NTU).

Accordingly, a transparent liquid cleansing composition as per the present invention (the composition) comprises:
a. a soap; and
b. an amphoteric surfactant,
wherein the soap and the amphoteric surfactant are present in weight ratio less than or equal to 2:1,
wherein the transparency of the composition is less than 100 NTU when measured at temperature in the range from 4 to 25 °C; and
wherein viscosity of the composition is in the range from 2,500 to 10,000 cps when measured at 25 °C.

### A Cleansing Composition

The composition is suitable for the purpose of cleansing a surface e.g. skin. Typically, the composition is used in suitable amount e.g. 5 to 10 mL; and applied to the skin using suitable means, often using hands, and alternatively sponge, loofah or similar along with copious amount of water to create lather; which is rinsed off with water to cleanse the surface, e.g. the skin.

### Soap

The composition comprises soap. Soap is salt of a fatty acid which also functions as an anionic surfactant. Soap when used in cleansing compositions, is known to improve the skin feel of the composition. However, soap, due to the alkaline pH, is often harsh on the skin possibly leading to dryness or itchy feel. Therefore, despite such preferred benefits soap may provide, using it in high amounts often leads to less preferred effects. Therefore, soap is used in moderate amounts in the present invention. However, even in moderate amounts like those described herein, presence of soap tends to cause opacity to liquid cleansing compositions. In such case, one way to obtain a transparent composition is to remove soap but this leads to loss of benefits delivered by soap. Therefore, delivering a transparent and appropriately viscous composition despite it contains soap remains a challenge. The present invention has solved this problem.

The soap is a salt of fatty acid that has carbon chain length from 6 to 30, preferably from 8 to 14 and more preferably from 10 to 12. Thus, sodium or potassium salts of fatty acids e.g. octanoic acid, caprylic acid, lauric acid, myristic acid and palmitic acid, are soaps that are particularly suitable for use in the present invention. Examples of soaps include potassium laurate, potassium myristate, potassium palmitate, sodium laurate, sodium myristate, sodium palmitate. In between sodium salts or potassium salts of fatty acids, potassium salts of fatty acids are preferred.

Preferably, the composition comprises from 0.01 to 7 wt %, more preferably from 0.05 to 6 wt %, even more preferably from 0.01 to 5 wt %, further more preferably from 0.05 to 4 wt %, still more preferably from 0.1 to 2 wt % and yet more preferably from 0.1 to 1 wt % soap.

### Amphoteric Surfactant

The composition further comprises an amphoteric surfactant. They provide foam boost and improve sensorial properties of the composition. Preferably, amphoteric surfactants are selected from betaine e.g. cocamidopropyl betaine (CAPB), sulfobetaine, cocoamphoacetate and mixtures thereof. More preferably, amophoteric surfactants are selected from betaine, sulfobetaine and mixtures thereof. Further more preferably, amphoteric surfactants selected is CAPB.

Preferably, the composition comprises from 1 to 5 wt %, more preferably from 1.5 to 4.5 wt %, even more preferably from 2 to 4 wt %, further more preferably from 2 to 3.5 wt % and still more preferably from 2.5 to 3 wt % amphoteric surfactants.

### Ratio of Soap and Amphoteric Surfactant

The composition comprises the soap and the amphoteric surfactant wherein the weight ratio of soap to amphoteric surfactant is less than or equal to 2:1. Preferably, the weight ratio of soap to amphoteric surfactant is less than 2:1. For example, if potassium laurate is the soap and CAPB is the amphoteric surfactant, then they are present in the weight ratio 2:1 or less than 2:1 e.g. 1.75:1, more preferably less than 1.5:1, even more preferably less than 1.25:1 and further more preferably 1:1. When the weight ratio of a soap and an amphoteric surfactant is more than 2:1, i.e. when it is not according to the present invention, it was found a liquid cleansing composition with required transparency and/or viscosity was not obtained.

It has been found that when the weight ratio of a soap and an amphoteric surfactant is as described above, a transparent and viscous liquid composition was obtained that exhibited transparency and viscosity as described below.

### Transparency

The composition exhibits transparency less than 100 NTU, preferably less than 75 NTU, more preferably less than 50 NTU, even more preferably less than 25 NTU, when measured at temperatures e.g. from 4 to 25 °C. Preferably, the composition is substantially isotropic and finely dispersed liquid cleansing composition. The phrase ‘substantially’, preferably means ‘more than 90%’, more preferably it means ‘more than 95%’ and even more preferably it means ‘more than 99%’, of the composition.

### Viscosity

The composition exhibits viscosity in the range 2,500 to 10,000 cps at 25 °C. Preferably, the composition exhibits viscosity in the range from 3,000 to 9,000 cps, more preferably from 3,500 to 8,500 cps, even more preferably from 4,000 to 8,000 cps and still more preferably from 4,000 to 7,000 cps, when measured at 25 °C. The viscosity throughout this specification has been reported in cps. In SI units, the conversion is that 1000 cps is one Pascal-second.

### Inorganic Salts

Preferably, the composition further comprises inorganic salts selected from sodium chloride, sodium iodide, sodium bromide, sodium sulfate, potassium chloride, potassium iodide, potassium bromide, potassium sulfate and mixtures thereof. More preferably, the inorganic salts are selected from sodium chloride, sodium sulfate, potassium chloride, potassium sulfate and mixtures thereof. Even more preferably, the inorganic salts are selected from sodium chloride, potassium chloride and mixtures thereof.

Preferably, the composition comprises from 0.01 to less than 3 wt %, more preferably from 0.05 to 2.5 wt %, even more preferably from 0.1 to 2 wt %, further more preferably from 0.5 to 1.5 wt % and still more preferably from 0.5 to 1 wt % of one or more inorganic salts described above.

### Non-Soap Anionic Surfactant

Preferably, the composition further comprises a non-soap anionic surfactant. Non-soap anionic surfactants such as those describe below, are known to provide foam and cleansing action.

Preferably, a non-soap anionic surfactants is selected from alkyl sulfates, alkyl ether sulfates (AES), alpha olefin sulfonates (AOS) and mixtures thereof. More preferably, the non-soap anionic surfactant is selected from alkyl sulfates, alkyl ether sulfates and mixtures thereof. Even more preferably, alkyl ether sulfates are used as the non-soap anionic surfactant in the composition.

Examples of alkyl sulfates that may be used as non-soap anionic surfactant in the composition include sodium lauryl sulfate (SLS), sodium myristyl sulfate; and mixtures thereof.

Examples of AES that may be used as non-soap anionic surfactants include anionic surfactants of the general formula:

R₁—(OR′)ₙ—O—SO₃⁻M⁺, wherein:
R₁ is saturated or unsaturated C₈-C₁₆, preferably C₁₂-C₁₄ alkyl chain; preferably, R₁ is a saturated C₈-C₁₆, more preferably a saturated C₁₂-C₁₄ alkyl chain;
R′ is ethylene; n is from 1 to 18; preferably from 1 to 15, more preferably from 1 to 10 and even more preferably from 1 to 5,
M⁺ is a suitable cation which provides charge neutrality, preferably sodium, calcium, potassium, or magnesium, more preferably a sodium cation.

Examples of AES that may be used as non-soap anionic surfactants include sodium lauryl ether sulfate (SLES), sodium myristyl ether sulfate and sodium palmityl ether sulfate and mixtures thereof. Preferred AES is SLES having 1 to 3 ethylene oxide units per molecule. SLES having 1 to 2 ethylene oxide units per molecule is more preferred.

Preferably, the composition comprises from 1 to 25 wt %, more preferably from 3 to 22 wt %, even more preferably from 5 to 18 wt %, further more preferably from 8 to 15 wt % and still more preferably from 10 to 12 wt % non-soap anionic surfactant that may be selected from one or more non-soap anionic surfactants described above.

### Octadecanoic Acid

Preferably, the composition further comprises an octadecanoic acid. It will be understood that the octadecanoic acid is an acid that contains 18 carbon atoms in a chain that may be linear or branched; and may be saturated or unsaturated at the same time.

Preferably, the octadecanoic acid is selected from 10-hydroxystearic acid (10-HSA), 12-HSA, petroselinic acid, conjugated linoleic acid and mixtures thereof. More preferably, the octadecanoic acid is selected from 10-HSA, 12-HSA, petroselinic acid and mixtures thereof. Even more preferably, the octadecanoic acid is selected from 10-HSA, 12-HSA and mixtures thereof. Further more preferably, the octadecanoic acid selected is 12-HSA.

The composition comprises an octadecanoic acid preferably from 0.01 to 2 wt %, more preferably from 0.05 to 1.5 wt %, even more preferably from 0.1 to 1 wt %, further more preferably from 0.25 to 0.75 wt % and still more preferably from 0.3 to 0.5 wt % octadecanoic acid.

### Polymers

Preferably, the composition does not comprise any viscosity modifying polymers. Preferably, the composition comprises 0 to 0.1 wt % viscosity modifying polymers.

However, if the composition comprises a viscosity modifying polymers they may be selected from water soluble/or dispersible polymers include the carbohydrate gums such as cellulose gum, microcrystalline cellulose, cellulose gel, hydroxyethyl cellulose, hydroxypropyl cellulose, hydroxypropyl methyl cellulose, sodium carboxymethylcellulose, methyl cellulose, ethyl cellulose, guar gum, gum karaya, gum tragacanth, gum arabic, gum acacia, gum agar, xanthan gum and mixtures thereof; modified and nonmodified starch granules and pregelatinized cold water soluble starch; emulsion polymers such as Aculyn® 28, Aculyn® 22 or Carbopol® Aqua SF1; cationic polymer such as modified polysaccharides including cationic guar available from Rhone Poulenc under the trade name Jaguar® C13S, Jaguar® C14S, Jaguar® C17, or Jaguar® C16; cationic modified cellulose such as UCARE® Polymer JR 30 or JR 40 from Amerchol; N-Hance® 3000, N-Hance® 3196, N-Hance® GPX 215 or N-Hance® GPX 196 from Hercules; synthetic cationic polymer such as Merquat® 100, Merquat® 280, Merquat® 281 and Merquat® 550 sold by Nalco; cationic starches such as StaLok® 100, 200, 300 and 400 sold by Staley Inc.; cationic galactomannans such as Galactasol® 800 series by Henkel, Inc.; Quadrosoft® LM-200; and Polyquaternium-24®. Also suitable are high molecular weight polyethylene glycols such as Polyox® WSR-205 (PEG 14M), Polyox® WSR-N-60K (PEG 45), and Polyox® WSR-301 (PEG 90M).

### pH of the Composition

Preferably, the pH of the composition is in the range from 8.0 to 9.5, more preferably, from 8.5 to 9.0.

### Water

Preferably, the composition comprises water from 60 to 98 wt %, more preferably from 65 to 95 wt %, even more preferably from 70 to 90 wt %, further more preferably from 75 to 85 wt % and still more preferably from 80 to 85 wt % water.

### Preservatives

Preferably, the composition further comprises preservatives to protect the composition against the growth of potentially harmful microorganisms. Particularly preferred preservatives include hydantoin derivatives, propionate salts, a variety of quaternary ammonium compounds, phenoxyethanol, imidazolidinyl urea, sodium dehydroacetate and benzyl alcohol. Preservatives are preferably used in amounts ranging from 0.01 wt % to 2 wt % and more preferably from 0.1 to 1 wt %.

### Emollients

The composition may further comprise emollients, such as stearyl alcohol, glyceryl monoricinoleate, mink oil, cetyl alcohol, isopropyl isostearate, stearic acid, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, di-n-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isostearic acid, palmitic acid, isopropyl linoleate, lauryl lactate, myristyl lactate, decyl oleate, myristyl myristate. Preferably, emollients are added in relatively low amount e.g. 0 to 3 wt % and 1 to 2 wt %.

### Optional Ingredients

The compositions may further comprise a wide range of other optional components e.g. antioxidants, buffering agents, colorants, astringents, perfume, humectants, conditioners, pH adjusters, skin soothing agents and skin healing agents.

The invention is described further using the following non-limiting examples.

### EXAMPLES

### Protocols:

### Preparation of Compositions:

All compositions shown in tables below, were made using deionized water. Water and potassium laurate powder (100%; Viva Corporation) were taken in amounts, as shown in tables below, in a container and mixed thoroughly using an overhead stirrer (Heidolph). Subsequently, the amphoteric surfactant, CAPB (30%; Galaxy surfactants), and a non-soap anionic surfactant, SLES (70%; Galaxy surfactants), were added to the container and were homogenized completely. Both these surfactants, SLES and CAPB, were taken in proportionate amount from their 70% and 30% concentrated stocks; respectively, so as to have the concentration of these surfactants as shown in the tables below. Lastly, salt (sodium chloride or potassium chloride; 100% lab grade) was added in amounts as shown in tables below.

### Measurement of Transparency

Once the compositions were made, it was stored at room temperature (~24±1 °C) overnight and the turbidity of the sample was measured using the turbidity meter using the following method. For this, a composition was filled in a glass vial, and it was ensured that air bubbles were not present. The glass vial was placed inside a turbidity meter (TURBIQUANTT 1100 IR) and measurements were carried out. The measurements were recorded once a stable reading was obtained. For measuring turbidity at lower temperatures, the compositions were stored in a refrigerator overnight, at 4±1 °C. The compositions were taken out from the refrigerator on the following day and turbidity measurement was carried out as describe above.

### Measurement of Viscosity

Zero calibration was done every time when using the viscometer (calibration without any spindle). The spindle RV-5 was fixed in the viscometer. The RPM and measurement time were set to 20 and 30 sec, respectively. The spindle was submerged into a test sample (a composition) until the mark mentioned on the spindle. As the measurement was started, the spindle rotated inside the sample and corresponding torque was measured and the instrument showed the calculated viscosity. The viscosity was measured after 30 sec; and was measured at room temperature, 25 °C. For this, Brookfield RV viscometer with RV-5 spindle, at 20 RPM for 30 sec was used.

### Examples 1 to 15 (According to the Invention) and Examples A to E (Control)

TABLE 1
Examples 1 to 10 according to the invention

| Ex. | 1 | 2 | 3 | 4 | 5 | 6 | 7 | 8 | 9 | 10 |
|---|---|---|---|---|---|---|---|---|---|---|
| Code | NCP 20 | NCP 22 | NCP 23 | NCP 27 | NCP 28 | NCP 29 | NCP 30 | NCP 31 | NCP 32 | NCP A |
| Total surf. | 15 | 10 | 10 | 12 | 12 | 12 | 10 | 15 | 10 | 7.8 |
| SLES | 9 | 6 | 5 | 7 | 6 | 5 | 4 | 5 | 4 | 5.3 |
| (on 100% | | | | | | | | | | |
| basis) | | | | | | | | | | |
| KLa | 3 | 1 | 2 | 2 | 3 | 4 | 3 | 5 | 3 | 1 |
| CAPB | 3 | 3 | 3 | 3 | 3 | 3 | 3 | 5 | 3 | 1.5 |
| (on 100% | | | | | | | | | | |
| basis) | | | | | | | | | | |
| KLa:CAPB | 1.0 | 0.3 | 0.7 | 0.7 | 1.0 | 1.3 | 1.0 | 1.0 | 1.0 | 0.7 |
| Salt | NaCl | NaCl | NaCl | NaCl | NaCl | NaCl | NaCl | NaCl | NaCl | NaCl |
| Salt, % | 0.925 | 0.62 | 1.19 | 1.38 | 1.67 | 2.34 | 1.95 | 1.41 | 2.2 | 1.1 |
| Minors e.g. | 1 | 1 | 1 | 1 | 1 | 1 | 1 | 1 | 1 | 1 |
| perfume | | | | | | | | | | |
| Water | To 100 | | | | | | | | | |
| Viscosity | 4980 | 7460 | 6520 | 4940 | 6700 | 4200 | 5380 | 8680 | 6060 | 5560 |
| pH | 8.7 | 8.06 | 8.46 | 8.56 | 8.78 | 8.74 | | | | 8.33 |
| Clarity at | clear | clear | clear | clear | clear | clear | clear | clear | clear | clear |
| 4 °C | | | | | | | | | | |
| (visual | | | | | | | | | | |
| observation) | | | | | | | | | | |
| NTU at 4 °C | | | | | | | | | 4.4 | |
| Clarity at | clear | clear | clear | clear | clear | clear | clear | clear | clear | clear |
| 25 °C | | | | | | | | | | |
| (visual | | | | | | | | | | |
| observation) | | | | | | | | | | |
| NTU at | | | | | | | | | 4.5 | |
| 25 °C | | | | | | | | | | |

(KLa: Potassium laurate)

TABLE 2
Examples 11 to 15 according to the invention

| Examples | 11 | 12 | 13 | 14 | 15 |
|---|---|---|---|---|---|
| Code | NCP 11 | NCP 18 | NCP 19 | NCP 25 | NCP 4.1 |
| Total surf | 15 | 15 | 12 | 12 | 15 |
| SLES | 12 | 10 | 7 | 9 | 8 |
| KLa | 2 | 3 | 3 | 2 | 4 |
| CAPB | 1 | 2 | 2 | 1 | 3 |
| KLa:CAPB | 2.0 | 1.5 | 1.5 | 2.0 | 1.3 |
| Salt | NaCl | NaCl | NaCl | NaCl | NaCl |
| Salt, % | 2 | 1.75 | 2.2 | 2.58 | 1.5 |
| Minor e.g. | 1 | 1 | 1 | 1 | 1 |
| perfume | | | | | |
| Water | To 100 | | | | |
| Viscosity | 9120 | 6100 | 4860 | 5520 | 6340 |
| pH | 8.7 | 8.79 | 8.7 | 8.82 | 8.71 |
| Clarity at 4 °C | clear | clear | clear | clear | clear |
| (visual | with a | with a | with a | with a | with a |
| observation) | few | few | few | few | few |
| | crystals | crystals | crystals | crystals | crystals |
| Clarity at 25 °C | clear | clear | clear | clear | clear |
| (visual | | | | | |
| observation) | | | | | |

(KLa: Potassium laurate)

TABLE 3
Control examples

| Examples | A | B | C | D | E |
|---|---|---|---|---|---|
| Code | NCP 12 | NCP 13 | NCP 26 | NCP 34 | NCP 34.1 |
| Total surf | 15 | 15 | 12 | 10 | 10 |
| SLES | 11 | 10 | 8 | 6 | 6 |
| KLa | 3 | 4 | 3 | 3 | 3 |
| CAPB | 1 | 1 | 1 | 1 | 1 |
| KLa:CAPB | 3.0 | 4.0 | 3.0 | 3.0 | 3.0 |
| Salt | NaCl | NaCl | NaCl | NaCl | NaCl |
| Salt, % | 2.15 | 3.1 | 3.4 | 5.24 | 0 |
| Minor e.g. | 1 | 1 | 1 | 1 | 1 |
| perfume | | | | | |
| Water | To 100 | | | | |
| Viscosity | 4160 | 4880 | 4620 | 1960 | 0 |
| pH | 8.84 | 8.94 | 8.82 | | |
| Clarity at 4 °C | precip | precip | precip | | precip |
| (visual | | | | | |
| observation) | | | | | |
| Clarity at 25 °C | clear | precip | precip | soft soap | clear |
| (visual | | | | | |
| observation) | | | | | |

(KLa: Potassium laurate; precip: precipitated matter observed)

### Examples 16, 17, F, G: Effect of Inclusion of Anionic Surfactant as Compared to Non-Ionic Surfactant

Compositions as shown in Table-4 below were prepared and the clarity (as observed visually) at the end of 24 hours was noted. Further the amount of foam generated was measured using the procedure as given below:

Compositions were diluted 100 times with deionized water (0.3 gm of product and 2.7 gm of water) in a graduated measuring cylinder (250 ml capacity). The measuring cylinder was closed with a glass lid and fixed inside an instrument for foam generation. The cylinders were rotated at 12.5 RPM for 40 rotations to generate foam. The average value of the foam generated from two cylinders is noted.

TABLE 4

| Ingredient | F | 16 | G | 17 |
|---|---|---|---|---|
| CAPB | 5.9 | 5.9 | 7.5 | 7.5 |
| Disodium coco | 2.0 | — | — | — |
| amphoacetate | | | | |
| Decyl glucoside | 3.0 | — | 6.0 | — |
| SLES | — | 3.0 | — | 6.0 |
| Lauric acid | 2.25 | 2.25 | — | — |
| Oleic acid | — | — | 1.5 | 1.5 |
| Potassium | 0.69 | 0.60 | 0.16 | 0.22 |
| hydroxide | | | | |
| Water | To 100 | To 100 | To 100 | To 100 |
| Clarity | Clear | Clear | Clear | Clear |
| Foam, mm (40 | 170 | 186 | 149 | 164 |
| rotations) | | | | |

The data in Table-4 above indicates that the composition as per the invention comprising anionic surfactant SLES (Examples 16 and 17) delivers better lather, with no compromise on clarity as compared to Examples outside the invention (Examples F and G) comprising non-ionic surfactant.

1. A transparent liquid cleansing composition comprising:
a. a soap; and
b. an amphoteric surfactant,
wherein the soap and the amphoteric surfactant are present in a weight ratio of less than or equal to 2:1,
wherein the transparency of the composition is less than 100 NTU when measured at temperature in the range from 4 to 25 °C; and
wherein viscosity of the composition is in the range from 2,500 to 10,000 cps when measured at 25 °C;
wherein the composition further comprises 1 to 25 wt % non-soap anionic surfactant selected from alkyl sulfate, alkyl ether sulfate, alpha olefin sulfonate and mixtures thereof;
wherein the pH of the composition is in the range from 8.0 to 9.5.

2. The composition as claimed in claim 1 wherein the composition comprises 0.01 to 7 wt % soap.

3. The composition as claimed in claim 1 wherein the soap is selected from salts of fatty acids having carbon chain length C₈ to C₁₄ and mixtures thereof.

4. The composition as claimed in claim 1 wherein the soap selected is from salts of fatty acids having carbon chain length C₁₀ to C₁₂ and mixtures thereof.

5. The composition as claimed in claim 1 wherein the soap is formed of potassium salt of a fatty acid.

6. The composition as claimed in claim 1 wherein the composition comprises from 1 to 5 wt % amphoteric surfactant.

7. The composition as claimed in claim 1 wherein the amphoteric surfactant is selected from betaine, sulfobetaine, cocoamphoacetate; and mixtures thereof.

8. The composition as claimed in claim 1 wherein the amphoteric surfactant is cocamidopropyl betaine.

9. The composition as claimed in claim 1 wherein the composition further comprises from 0.1 to less than 3 wt % inorganic salt selected from sodium chloride, sodium iodide, sodium bromide, sodium sulfate, potassium chloride, potassium iodide, potassium bromide, potassium sulfate; and mixtures thereof.

10. The composition as claimed in claim 1 wherein the composition further comprises an octadecanoic acid selected from 10-hydroxystearic acid, 12-hydroxystearic acid, petroselinic acid, conjugated linoleic acid and mixtures thereof.

11. The composition as claimed in claim 10 wherein the octadecanoic acid is selected from 10-hydroxystearic acid, 12-hydroxystearic acid and mixtures thereof.

12. The composition as claimed in claim 11 wherein the octadecanoic acid is 12-hydroxystearic acid.

13. (canceled)
